(19) United States
(12) Patent Application Publication
(10) Pub. No.: US 2022/0347384 A1
(43) Pub. Date: Nov. 3, 2022

(54) WIRED CONTINUOUS GLUCOSE MONITORING INFUSION SET

(71) Applicant: Pacific Diabetes Technologies Inc, Portland, OR (US)

(72) Inventors: Kenneth Hillen, Beaverton, OR (US); Thomas L. Seidl, Wilsonville, OR (US); Solomon Reid, Portland, OR (US); William Kenneth Ward, Portland, OR (US)

(21) Appl. No.: 17/730,745

(22) Filed: Apr. 27, 2022

Related U.S. Application Data
(60) Provisional application No. 63/181,057, filed on Apr. 28, 2021.

(51) Int. Cl. A61M 5/168; A61B 5/145; A61M 5/142; A61M 5/50

(52) U.S. Cl. CPC A61M 5/16836; A61B 5/14532; A61M 5/142; A61M 5/5086; A61M 2205/13

(57) ABSTRACT

The present disclosure provides systems and methods for managing blood glucose in a subject. An insulin delivery cannula may comprise a hollow tube comprising proximal and distal ends, the proximal end in fluid communication with an insulin source, and the distal end configured to deliver insulin into a subcutaneous space. An insulin infusion pump may be fluidically coupled to the proximal end, and attached to the insulin delivery cannula directly or via an intervening tube. The glucose sensor and the insulin delivery cannula may be configured to be inserted into the subcutaneous space simultaneously by a single insertion device. A lumen of the insulin delivery cannula may be contiguous with a lumen of tubing from the insulin infusion pump. Electrical conductors for the electrodes may be disposed on a wall of the tubing or attached to a surface of the tubing, and establish an electrical connection with an electronic module.

### CROSS-REFERENCE

This application claims the benefit of U.S. Provisional Patent Application No. 63/181,057, filed Apr. 28, 2021, which is incorporated by reference herein in its entirety.

### BACKGROUND

Many continuous glucose monitoring (CGM)/tubed-insulin pump integrations may use a separate electronic module to wirelessly send glucose readings to the pump. The result is that diabetes management benefiting from both insulin delivery and glucose measurement may require the use of 3 separate devices: pump, infusion set and CGM. Such a CGM configuration may also comprise a battery and electronics, resulting in a larger, more costly device. The wireless communications may use the unlicensed (Industrial-Scientific-Medical) (ISM) band which may be subject to interference from many other users of this band. The separate CGM sensor and insulin infusion cannula may both penetrate the skin, resulting in increased diabetes patient discomfort.

### SUMMARY

The present disclosure provides devices, systems, and methods in which the CGM sensor and insulin infusion site may be combined into a small single device connected to the insulin pump using tubing and wires (W-CGMIS, wired CGM infusion set). The necessary wires can be embedded in the walls of the insulin tubing or bonded securely to it. The detachable tubing connections at the CGMIS and pump may also have electrical connections to simplify disconnection. In one embodiment, the CGMIS contains minimal collocated electronics to minimize low level analog signal issues. In one embodiment, the additional space along the infusion tube is used for additional signaling conductors that enable additional safety features.

The W-CGMIS may be a simple, inexpensive alternative to the CGMIS to create one device that offers both insulin delivery and glucose measurement.

In addition to the device simplicity, the wired connection of the devices, systems, and methods of the present disclosure may provide several other benefits. These include the minimization of interference from nearby ISM band radios, lowering the cost of the CGM/pump system, eliminating electronics and batteries that need to be recycled or properly disposed of, and the addition of safety features such as tubing disconnection detection.

In an aspect, the present disclosure provides a system for managing blood glucose in a subject, comprising: an insulin delivery cannula comprising a hollow tube comprising a proximal end and a distal end, wherein the proximal end is in fluid communication with a source of an insulin or insulin analog formulation, wherein the distal end is configured to deliver the insulin or insulin analog formulation into a subcutaneous space of the subject; a glucose sensor located no more than a pre-determined distance away from the distal end, wherein the glucose sensor is a continuous amperometric or coulometric glucose sensor comprising an indicating electrode and a reference electrode; and an insulin infusion pump fluidically coupled to the proximal end of the insulin delivery cannula, wherein the insulin infusion pump is attached to the insulin delivery cannula either directly or via an intervening tube, wherein the glucose sensor and the insulin delivery cannula are configured to be inserted into the subcutaneous space of the subject simultaneously by a single insertion device, wherein a lumen of the insulin delivery cannula is contiguous with a lumen of tubing that emerges from the insulin infusion pump, wherein electrical conductors for the indicating electrode and the reference electrode are disposed on a wall of the tubing or attached to a surface of the tubing, and establish an electrical connection with an electronic module located within a housing that encloses the insulin infusion pump.

In some embodiments, the insulin or insulin analog formulation comprises an excipient comprising a phenolic compound (e.g., phenol) or cresol. In some embodiments, the excipient comprises the phenolic compound. In some embodiments, the excipient comprises the cresol.

In some embodiments, the housing comprises an upper accessible surface and a lower surface configured to be adhered to a skin surface of the subject.

In some embodiments, the indicating electrode comprises gold, carbon, graphite, platinum, or iridium.

In some embodiments, the pre-determined distance is about 15 millimeters (mm), about 14 mm, about 13 mm, about 12 mm, about 10 mm, about 9 mm, about 8 mm, about 7 mm, about 6 mm, about 5 mm, about 4.5 mm, about 4 mm, about 3.5 mm, about 3 mm, about 2.5 mm, about 2 mm, about 1.5 mm, or about 1 mm. In some embodiments, the pre-determined distance is about 7 mm. In some embodiments, the pre-determined distance is about 5 mm. In some embodiments, the pre-determined distance is about 2.5 mm.

In some embodiments, the glucose sensor comprises an electrode layer comprising the indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase. In some embodiments, the ligand is pyridine-based. In some embodiments, the ligand is 4,4′-dimethyl-2,2′-bipyridine. In some embodiments, the ligand is imidazole-based. In some embodiments, the redox mediator is bound to poly (4-vinyl pyridine). In some embodiments, the redox mediator is bound to poly (1-vinyl imidazole).

In some embodiments, the glucose sensor further comprises an insulating layer and a metal layer, wherein the insulating layer is coupled to the metal layer, and wherein the metal layer is coupled to the electrode layer. In some embodiments, the insulating layer comprises a polyimide or liquid crystal polymer. In some embodiments, the metal layer has a thickness of at least about 1 micrometer (μm), 2 μm, 3 μm, 4 μm, 5 μm, 6 μm, 7 μm, 8 μm, 9 μm, or 10 μm. In some embodiments, the metal layer comprises titanium, gold, or platinum. In some embodiments, the electrode layer comprises a film having a thickness of no more than about 1000 nanometers (nm), 900 nm, 800 nm, 700 nm, 600 nm, 500 nm, 400 nm, 300 nm, 200 nm, or 100 nm.

In some embodiments, the redox mediator and the enzyme allow electron transfer from subcutaneous glucose to the indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode. In some embodiments, the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the insulin or insulin analog formulation.

In some embodiments, the metal compound comprises a metal selected from the group consisting of: Osmium, Ruthenium, Palladium, Platinum, Rhodium, Iridium, Cobalt, Iron, and Copper. In some embodiments, the metal compound comprises a metal selected from the group consisting of: Ruthenium, Palladium, Platinum, Rhodium, Iridium, Cobalt, Iron, and Copper. In some embodiments, the ligand comprises a heterocyclic nitrogen compound, a pyridine ring combined with an imidazole ring, a non-nitrogen element substituted into a heterocycle ring, or an accessory “R” group bound to a heterocyclic ring. In some embodiments, the heterocyclic nitrogen compound comprises pyridine or imidazole with 1 ring, 2 rings, 3 rings, or 4 rings.

In some embodiments, the reference electrode is a combined reference/counter electrode. In some embodiments, the reference electrode comprises a silver/silver chloride (Ag/AgCl) reference electrode.

In some embodiments, the glucose sensor is disposed on an outer wall of the hollow tube. In some embodiments, the glucose sensor is the continuous amperometric glucose sensor. In some embodiments, the glucose sensor is the continuous coulometric glucose sensor.

In some embodiments, the system further comprises a cartridge comprising the source of the insulin or insulin analog formulation. In some embodiments, the reference electrode is a combined counter- and reference electrode.

In some embodiments, the electrical conductors for the indicating electrode and the reference electrode are disposed on the wall of the tubing. In some embodiments, the electrical conductors for the indicating electrode and the reference electrode are attached to an inner surface of the tubing. In some embodiments, the electrical conductors for the indicating electrode and the reference electrode are attached to an outer surface of the tubing.

In some embodiments, the system further comprises a transimpedance amplifier (TIA) operably connected to the glucose sensor, wherein the TIA is configured to convert an electrical current generated by the glucose sensor to a voltage value. In some embodiments, the voltage value is indicative of a blood glucose level of the subject. In some embodiments, the TIA is located within the electronic module.

In another aspect, the present disclosure provides a method for managing blood glucose in a subject, comprising: (a) obtaining a device for delivery of the insulin or insulin analog formulation and measurement of subcutaneous glucose concentration, wherein the device comprises: (i) a hollow tube comprising a proximal end and a distal end, wherein the proximal end is in fluid communication with a source of an insulin or insulin analog formulation, wherein the distal end is configured to deliver the insulin or insulin analog formulation into a subcutaneous space of the subject; (ii) a glucose sensor located no more than a pre-determined distance away from the distal end, wherein the glucose sensor is a continuous amperometric or coulometric glucose sensor comprising an indicating electrode and a reference electrode; and (iii) an insulin infusion pump fluidically coupled to the proximal end of the insulin delivery cannula, wherein the insulin infusion pump is attached to the insulin delivery cannula either directly or via an intervening tube, wherein a lumen of the insulin delivery cannula is contiguous with a lumen of tubing that emerges from the insulin infusion pump, wherein electrical conductors for the indicating electrode and the reference electrode are disposed on a wall of the tubing or attached to a surface of the tubing; (b) connecting the proximal end of the hollow tube to the source of the concentrated insulin or insulin analog formulation; (c) establishing an electrical connection with an electronic module located within a housing that encloses the insulin infusion pump; (d) inserting the glucose sensor and the distal end of the hollow tube of the insulin delivery cannula into the subcutaneous space of the subject simultaneously by a single insertion device; and (e) simultaneously (1) delivering the insulin or insulin analog formulation into a subcutaneous space of the subject using the insulin infusion pump, and (2) measuring the subcutaneous glucose concentration of the subject.

In some embodiments, the insulin or insulin analog formulation comprises an excipient comprising a phenolic compound (e.g., phenol) or cresol. In some embodiments, the excipient comprises the phenolic compound. In some embodiments, the excipient comprises the cresol.

In some embodiments, the housing comprises an upper accessible surface and a lower surface configured to be adhered to a skin surface of the subject.

In some embodiments, the indicating electrode comprises gold, carbon, graphite, platinum, or iridium.

In some embodiments, the pre-determined distance is about 15 millimeters (mm), about 14 mm, about 13 mm, about 12 mm, about 10 mm, about 9 mm, about 8 mm, about 7 mm, about 6 mm, about 5 mm, about 4.5 mm, about 4 mm, about 3.5 mm, about 3 mm, about 2.5 mm, about 2 mm, about 1.5 mm, or about 1 mm. In some embodiments, the pre-determined distance is about 7 mm. In some embodiments, the pre-determined distance is about 5 mm. In some embodiments, the pre-determined distance is about 2.5 mm.

In some embodiments, the glucose sensor comprises an electrode layer comprising the indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase. In some embodiments, the ligand is pyridine-based. In some embodiments, the ligand is 4,4′-dimethyl-2,2′-bipyridine. In some embodiments, the ligand is imidazole-based. In some embodiments, the redox mediator is bound to poly (4-vinyl pyridine). In some embodiments, the redox mediator is bound to poly (1-vinyl imidazole).

In some embodiments, the glucose sensor further comprises an insulating layer and a metal layer, wherein the insulating layer is coupled to the metal layer, and wherein the metal layer is coupled to the electrode layer. In some embodiments, the insulating layer comprises a polyimide or liquid crystal polymer. In some embodiments, the metal layer has a thickness of at least about 1 micrometer (μm), 2 μm, 3 μm, 4 μm, 5 μm, 6 μm, 7 μm, 8 μm, 9 μm, or 10 μm. In some embodiments, the metal layer comprises titanium, gold, or platinum. In some embodiments, the electrode layer comprises a film having a thickness of no more than about 1000 nanometers (nm), 900 nm, 800 nm, 700 nm, 600 nm, 500 nm, 400 nm, 300 nm, 200 nm, or 100 nm.

In some embodiments, the redox mediator and the enzyme allow electron transfer from subcutaneous glucose to the indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode. In some embodiments, the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the insulin or insulin analog formulation.

In some embodiments, the metal compound comprises a metal selected from the group consisting of: Osmium, Ruthenium, Palladium, Platinum, Rhodium, Iridium, Cobalt, Iron, and Copper. In some embodiments, the metal compound comprises a metal selected from the group consisting of: Ruthenium, Palladium, Platinum, Rhodium, Iridium, Cobalt, Iron, and Copper. In some embodiments, the ligand comprises a heterocyclic nitrogen compound, a pyridine ring combined with an imidazole ring, a non-nitrogen element substituted into a heterocycle ring, or an accessory “R” group bound to a heterocyclic ring. In some embodiments, the heterocyclic nitrogen compound comprises pyridine or imidazole with 1 ring, 2 rings, 3 rings, or 4 rings.

In some embodiments, the reference electrode is a combined reference/counter electrode. In some embodiments, the reference electrode comprises a silver/silver chloride (Ag/AgCl) reference electrode.

In some embodiments, the glucose sensor is disposed on an outer wall of the hollow tube. In some embodiments, the glucose sensor is the continuous amperometric glucose sensor. In some embodiments, the glucose sensor is the continuous coulometric glucose sensor.

In some embodiments, the method further comprises establishing fluid communication between the proximal end and a cartridge comprising the source of the concentrated insulin or insulin analog formulation. In some embodiments, the reference electrode is a combined counter- and reference electrode.

In some embodiments, the electrical conductors for the indicating electrode and the reference electrode are disposed on the wall of the tubing. In some embodiments, the electrical conductors for the indicating electrode and the reference electrode are attached to an inner surface of the tubing. In some embodiments, the electrical conductors for the indicating electrode and the reference electrode are attached to an outer surface of the tubing.

In some embodiments, the system further comprises a transimpedance amplifier (TIA) operably connected to the glucose sensor, wherein the TIA is configured to convert an electrical current generated by the glucose sensor to a voltage value. In some embodiments, the voltage value is indicative of a blood glucose level of the subject. In some embodiments, the TIA is located within the electronic module.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

Embodiments will be readily understood by the following detailed description in conjunction with the accompanying drawings and the appended claims. Embodiments are illustrated by way of example and not by way of limitation in the figures of the accompanying drawings.

FIGS. 1A-1D illustrate an example of a glucose sensing cannula, housing & connector—passive with filter. These figures shows a simple configuration for connecting the CGMIS to a pump simply using tubing and wires. In this embodiment, the sensor is connected to the same sensor circuitry, being separated from it by two wires. The circuitry is located in the insulin pump at the other end of the insulin tubing. The sensor direct current (DC) bias voltage is applied across the wires and the sensor response current flows on the same wires. The current is converted to a voltage by a transimpedance amplifier (TIA).

This wiring may be subject to potential interference from electromagnetic signals. This interference can be mitigated using low pass filters, twisted pair wires, or both. In one embodiment, the filter topology is a “T” filter which comprises series ferrite beads and a shunt capacitor. A similar filter may be used at each end of the wires. The passive circuitry can also include a bulk storage capacitor to maintain the bias voltage to the sensor during momentary cable disconnects.

FIG. 1A illustrates an overview image of said implementation with the connector and tubing.

FIG. 1B illustrates a cut-away view of the implementation. The fluidic and electrical connections from the sensing cannula to the electro-fluidic connector are shown, with the various components labeled.

FIG. 1C illustrates the tubing and electro-fluidic connector.

FIG. 1D illustrates the filter network used for lab testing. Two different ferrite compounds are used to cover a wider range of frequencies. The capacitor may be a low leakage capacitor to prevent sensor errors.

FIG. 2 illustrates an example of a glucose sensing cannula, housing, PCB & connector—active. The figure shows another variation which has some active electronic circuitry embedded with the sensor. In one embodiment, this circuitry comprises a TIA to convert the sensor response current to a voltage to further reduce sensitivity to interference, a voltage source to supply the IE (indicating electrode) bias voltage and some low pass filter components. The circuitry can also include a bulk storage capacitor to maintain the bias voltage to the sensor during momentary cable disconnects. This variation includes 4 wires, which provide power for the amplifier, supply the sensor bias voltage from the attached pump/readout device, and provide a path for the sensor response voltage to be transmitted to the pump/readout device.

An alternative implementation may include the use a current amplifier to convert the high impedance low level sensor output current into larger current from a low impedance source, further reducing susceptibility to interference and eliminating potential error caused by voltage drop in the wiring.

FIG. 3 illustrates an example of tubing with 1-2 wires running in parallel down opposite sides of tube. There are numerous ways to manufacture a tube with conductors. FIG. 3 shows insulated wires bonded to the sides of the tube. This bonding may be implemented using thermal methods, chemical methods, or by the use of adhesives. In one embodiment, the connector is fixed to the tubing/wires using injection molding to create a robust attachment. Insulin pump tubing may be flexible and capable of some stretching. To maintain these desirable characteristics, various methods can be employed. These include using wires wound like a spring, using printable “liquid” wire, etc.

FIG. 4 illustrates an example of tubing with 2-2 wires twisted on one side of tube. Twisted pair wires may be used to reduce susceptibility to electromagnetic interference. In this case, twisted pair wires can also be bonded to or embedded in the wall of the insulin tubing. If twisted pair wiring is used, the wire pair may be located on one side of the tube. This figure shows the wires embedded in the wall of the tube. If 4 wires are used in the wired CGMIS system, one twisted wire pair may be placed on each side of the tube. This embedding can be done using standard extrusion processes.

FIG. 5 illustrates an example of tubing with 3-4 wires 2 parallel on each side of tube. This example shows the 4 wires embedded in the walls of the tube.

FIG. 6 illustrates an example of a tubing/cartridge/pump interface. The wired CGMIS may need connections to an insulin pump. These connections can be made a number of ways depending on the pump reservoir construction. FIG. 6 shows one possible implementation. In this implementation, the same electro-fluidic connector is used to connect to the pump insulin cartridge. The cartridge may have electrical connections to the body of the pump along with its normal mechanical connections for insulin dispensing. Other electro-fluidic connectors can also be used, providing flexibility to the pump manufacturers.

FIG. 7 illustrates an example of tubing/cartridge/pump interface. FIG. 7 shows a variation of FIG. 6 where the tubing and wires are permanently connected to the pump cartridge. Due to the size of the connector, some pump manufacturers may alternatively permanently connect the tuning/wires to the pump cartridge,

FIG. 8 illustrates an example of tubing/cartridge/pump interface. FIG. 8 shows the tubing with two connectors at the pump end, one electrical connector that connects directly to the pump and the other fluidic connector that connects to the insulin reservoir. This approach may simplify the addition of the W-CGMIS to a pump without redesigning its insulin reservoir. An electrical connection may be added elsewhere on the pump body.

### DETAILED DESCRIPTION

The patent or application file contains at least one drawing executed in color. Copies of this patent or patent application publication with color drawing(s) will be provided by the Office upon request and payment of the necessary fee.

References are made herein to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

As used herein, the term “cannula” or “insulin delivery cannula” generally refers to a hollow tube (e.g., round, flattened, or other in cross section) that terminates in the subcutaneous space of a human and is intended to serve as a conduit through which insulin or insulin analog can be infused or injected. The cannula may be fabricated using a rigid material, such as a polymer or a metal, having an interior (e.g., inner) surface and an exterior (e.g., outer) surface, and an opening at both ends.

As used herein, the term “sensing cannula” generally refers to a cannula having an analyte sensor disposed on the exterior surface and one or more fluid delivery channels contained within the cannula.

As used herein, the term “continuous glucose monitor (CGM)” generally refers to a system comprising electronics configured for continuous or nearly continuous measurement of glucose levels from a subject (e.g., a human being, an animal, or a mammal) and/or reporting of such measurements.

As used herein, the term “CGM injection port” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as an insulin pen, a syringe, or another fluid delivery device.

As used herein, the term “CGM infusion set” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as a pump or a gravity-fed sourced source.

The terms “coupled” and “connected,” along with their derivatives, may be used herein. It should be understood that these terms are not intended as synonyms for each other. Rather, in particular embodiments, “connected” may be used to indicate that two or more elements are in direct physical or electrical contact with each other. “Coupled” may be used to indicate that two or more elements are in direct physical or electrical contact. However, “coupled” may also be used to indicate that two or more elements are not in direct contact with each other, but yet still cooperate or interact with each other.

As used herein, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and C). For the purposes of the description, a phrase in the form “(A)B” means (B) or (AB) that is, A is an optional element.

As used herein, the terms “embodiment” or “embodiments,” may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, the plural can be translated to the singular and/or the singular can be translated to the plural, as is appropriate to the context and/or application. The various singular/plural permutations may be expressly set forth herein for sake of clarity.

Many continuous glucose monitoring (CGM) devices and systems may use an electronic module connected to the CGM sensor and attached to the skin directly over it. For example, a Bluetooth radio operating in the unlicensed 2.4 GHz Industrial-Scientific-Medical (ISM) band may be used to communicate with an insulin pump or display device. The ISM band is often over-subscribed which results in interference and lost data. The electronic module may comprise a battery, electronic circuitry, microprocessor and a Bluetooth radio and its associated antenna. This electronic module may be large and may be often visible underneath tight-fitting clothing. Also, it may be prone to catching on table edges, door jambs, etc. often dislodging the sensor and necessitating its replacement at an additional cost. The average lifetime of a CGM/CGMIS electronics module may be about 6 months. At the end of its life, it may need to be discarded, or it may need to be recycled depending on the battery used and local rules, resulting in an additional burden on the user. Closed loop systems may require that the pump and receiver be located near the CGM transmitter to minimize the risk of interference of the Bluetooth connection by the patient's body.

In some cases, the tubing connected to an insulin infusion site can become disconnected if the tubing is pulled with sufficient force. In other cases, the infusion site may become dislodged if the force on the tubing is applied in a different direction. This undesirable condition may go unobserved by the user until their glucose value starts rinsing inexplicably.

The present disclosure provides device, systems, and methods that may replace the traditional wireless connection between the CGM/CGMIS and pump/readout device with a wired connection. Such device, systems, and methods of the present disclosure may take advantage of the existing tubing tethering the pump to the infusion site and include wires either situated beside the tubing or attached to the tubing. This configuration may eliminate the need for a microprocessor, wireless communication circuitry (e.g., Bluetooth or WiFi radio and its antenna) and the battery from the CGM/CGMIS, and may situate the sensor bias and signal conditioning electronics in the insulin pump/display device.

Eliminating the battery and wireless communication circuitry from the disposable element may provide a lower cost and more environmentally-conscious product. Without the battery and circuitry, the skin-attached portion of the CGM/CGMIS may be much smaller and have a lower profile, making it less visible under clothing. The replacement of the Bluetooth connection with a wired connection may significantly reduce or virtually eliminate the possibility of interference and allow the pump/display device to be located anywhere the tubing length permits.

Additional features can be supported using either more wires or signal multiplexing if desired. These include device disconnection detection, dislodgement detection, etc. CGM/CGMIS disconnection may be detected by observing the higher impedance or loss of current flowing through the sensor. CGMIS dislodgement may be detected by observing an abrupt change in the current flow through the sensor.

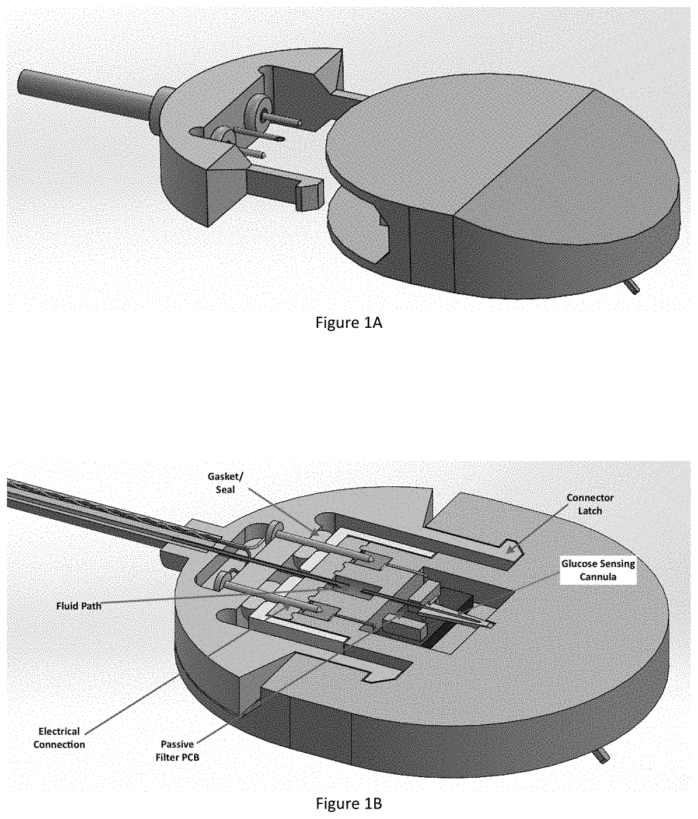

FIGS. 1A-1D illustrate an example of a glucose sensing cannula, housing & connector—passive with filter. These figures shows a simple configuration for connecting the CGMIS to a pump simply using tubing and wires. In this embodiment, the sensor is connected to the same sensor circuitry, being separated from it by two wires. The circuitry is located in the insulin pump at the other end of the insulin tubing. The sensor direct current (DC) bias voltage is applied across the wires and the sensor response current flows on the same wires. The current is converted to a voltage by a transimpedance amplifier (TIA).

This wiring may be subject to potential interference from electromagnetic signals. This interference can be mitigated using low pass filters, twisted pair wires, or both. In one embodiment, the filter topology is a “T” filter which comprises series ferrite beads and a shunt capacitor. A similar filter may be used at each end of the wires. The passive circuitry can also include a bulk storage capacitor to maintain the bias voltage to the sensor during momentary cable disconnects.

FIG. 1A illustrates an overview image of said implementation with the connector and tubing.

FIG. 1B illustrates a cut-away view of the implementation. The fluidic and electrical connections from the sensing cannula to the electro-fluidic connector are shown, with the various components labeled.

FIG. 1C illustrates the tubing and electro-fluidic connector.

FIG. 1D illustrates the filter network used for lab testing. Two different ferrite compounds are used to cover a wider range of frequencies. The capacitor may be a low leakage capacitor to prevent sensor errors.

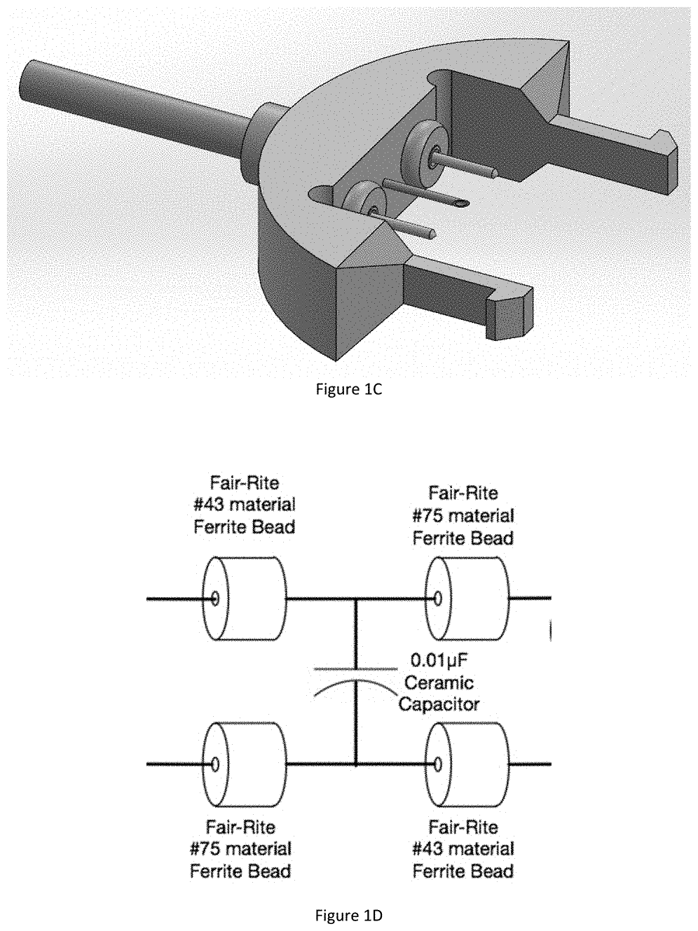

FIG. 2 illustrates an example of a glucose sensing cannula, housing, PCB & connector—active. The figure shows another variation which has some active electronic circuitry embedded with the sensor. In one embodiment, this circuitry comprises a TIA to convert the sensor response current to a voltage to further reduce sensitivity to interference, a voltage source to supply the IE (indicating electrode) bias voltage and some low pass filter components. The circuitry can also include a bulk storage capacitor to maintain the bias voltage to the sensor during momentary cable disconnects. This variation includes 4 wires, which provide power for the amplifier, supply the sensor bias voltage from the attached pump/readout device, and provide a path for the sensor response voltage to be transmitted to the pump/readout device.

An alternative implementation may include the use of a current amplifier to convert the high impedance low level sensor output current into larger current from a low impedance source, further reducing susceptibility to interference.

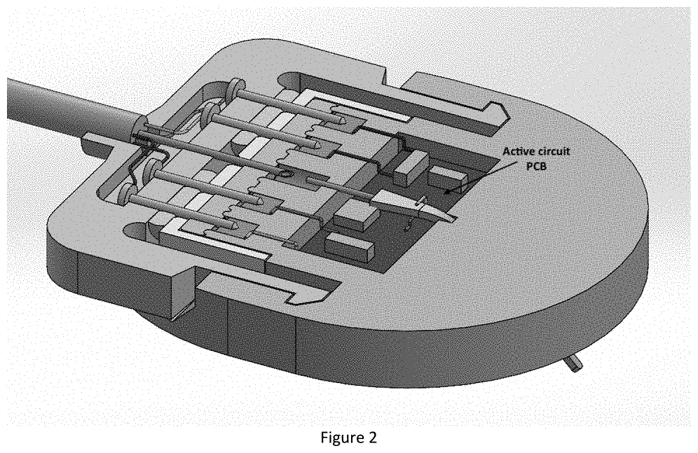

FIG. 3 illustrates an example of tubing with 1-2 wires running in parallel down opposite sides of tube. There are numerous ways to manufacture a tube with conductors. FIG. 3 shows insulated wires bonded to the sides of the tube. This bonding may be implemented using thermal methods, chemical methods, or by the use of adhesives. In one embodiment, the connector is fixed to the tubing/wires using injection molding to create a robust attachment. Insulin pump tubing may be flexible and capable of some stretching. To maintain these desirable characteristics, various methods can be employed. These include using wires wound like a spring, using printable “liquid” wire, etc.

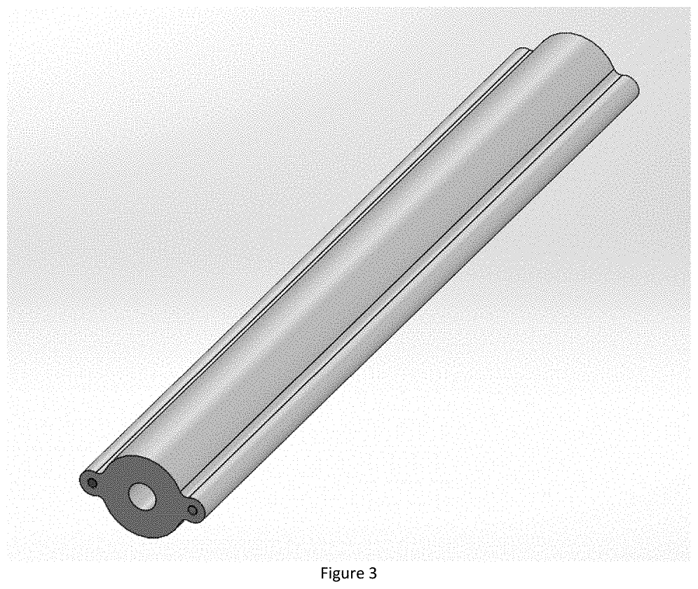

FIG. 4 illustrates an example of tubing with 2-2 wires twisted on one side of tube. Twisted pair wires may be used to reduce susceptibility to electromagnetic interference. In this case, twisted pair wires can also be bonded to or embedded in the wall of the insulin tubing. If twisted pair wiring is used, the wire pair may be located on one side of the tube. This figure shows the wires embedded in the wall of the tube. If 4 wires are used in the wired CGMIS system, one twisted wire pair may be placed on each side of the tube. This embedding can be done using standard extrusion processes.

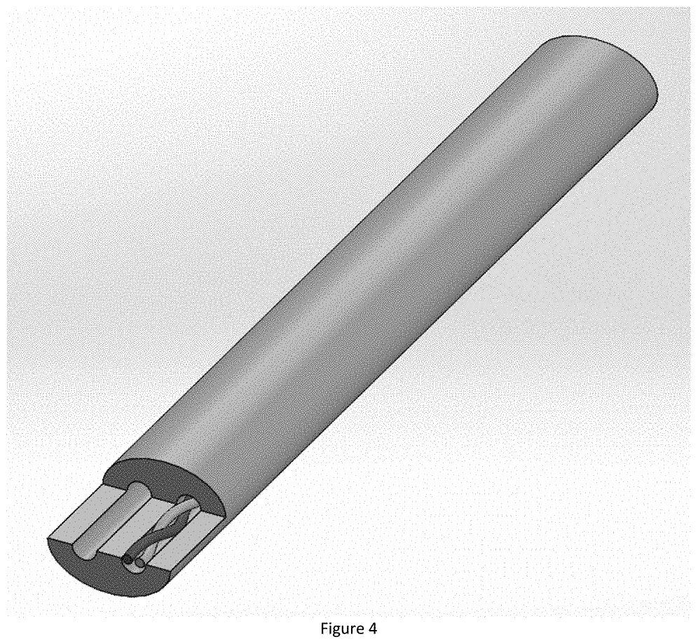

FIG. 5 illustrates an example of tubing with 3-4 wires 2 parallel on each side of tube. This example shows the 4 wires embedded in the walls of the tube.

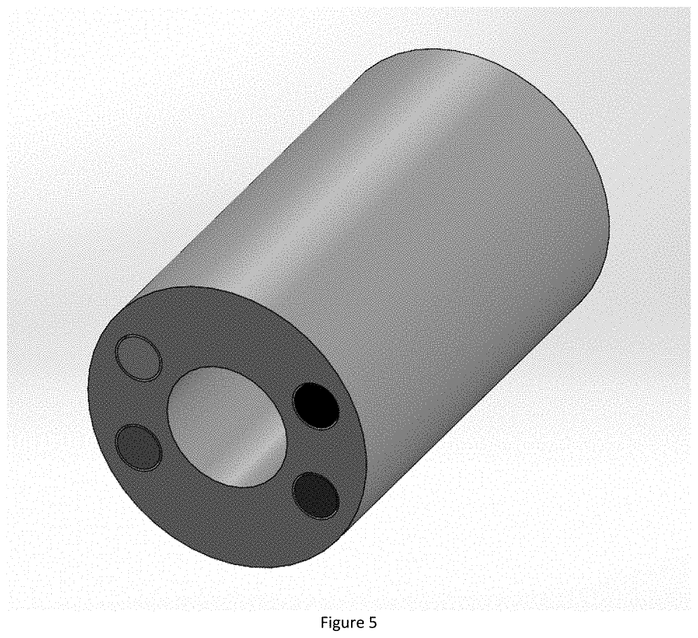

FIG. 6 illustrates an example of a tubing/cartridge/pump interface. The wired CGMIS may need connections to an insulin pump. These connections can be made a number of ways depending on the pump reservoir construction. FIG. 6 shows one possible implementation. In this implementation, the same electro-fluidic connector is used to connect to the pump insulin cartridge. The cartridge may have electrical connections to the body of the pump along with its normal mechanical connections for insulin dispensing. Other electro-fluidic connectors can also be used, providing flexibility to the pump manufacturers.

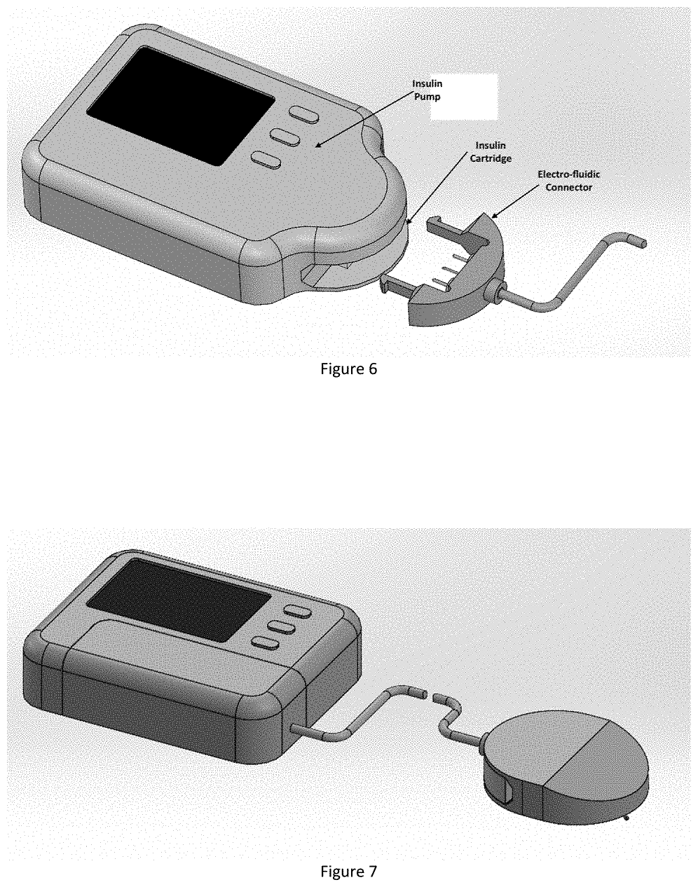

FIG. 7 illustrates an example of tubing/cartridge/pump interface. FIG. 7 shows a variation of FIG. 6 where the tubing and wires are permanently connected to the pump cartridge. Due to the size of the connector, some pump manufacturers may alternatively permanently connect the tuning/wires to the pump cartridge,

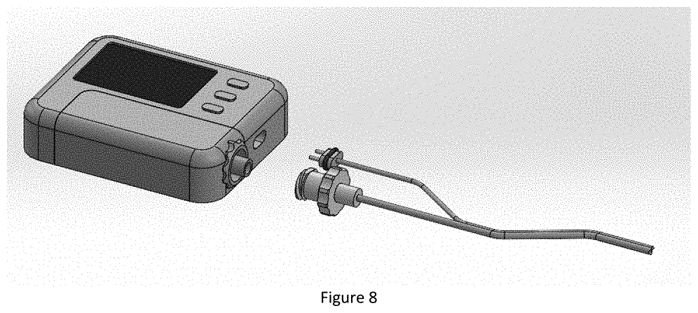

FIG. 8 illustrates an example of tubing/cartridge/pump interface. FIG. 8 shows the tubing with two connectors at the pump end, one electrical connector that connects directly to the pump and the other fluidic connector that connects to the insulin reservoir. This approach may simplify the addition of the W-CGMIS to a pump without redesigning its insulin reservoir. An electrical connection may be added elsewhere on the pump body.

Fabrication of Glucose-Sensing Elements (e.g., in the Outer Wall of an Insulin Delivery Cannula)

In order to minimize the interference by reducing the bias potential, a redox-mediated chemistry scheme may be used. For example, the specific use of low bias, redox-based sensing technology to avoid preservative artifact is described in U.S. Pat. No. 10,780,222 (Ward et al), which is incorporated by reference herein in its entirety.

Osmium, ruthenium, and other compounds may be suitable for accepting electrons from glucose oxidase, more specifically from the prosthetic group of glucose oxidase known as flavin adenine dinucleotide (FAD). In one embodiment, the osmium is held in placed (coordinated) by its bond to a ligand such as, 4,4′-dimethyl 2,2′-bipyridine. Many other ligands can be used. Various electron rich chemical groups such as methyl, methoxy or amino, when bound to the pyridine or imidazole ligands, may allow the osmium to transfer electrons at a low polarizing bias. In some cases, the ligands, which coordinate, and bind to, the osmium redox centers, may be bound to a polymer, and this complex of mediator, ligand and polymer may be referred to as the redox mediator polymer (RMP).

In one embodiment, osmium is the redox mediator. The polymer backbone of the RMP comprises poly(l-vinyl imidazole) (PVI). Two coordination ligands, 4,4′-dimethyl,2,2′-bipyridine may be bound to an osmium group. The osmium may be bound to approximately one of every 5 to 15 imidazole groups on the PVI.

In one embodiment, the RMP is deposited on a gold indicating electrode, but other metals can be used, such as vitreous carbon, glassy carbon, graphite, platinum, or iridium. It is also possible to make the indicating electrode porous, for example by the use of acid anodization, laser poration, or plasma etching.

The enzyme can be glucose oxidase or glucose dehydrogenase, and is in contact with the osmium or other mediator, the ligand and the RMP. The enzyme can be extended by the use of albumin, collagen carrageenan, or other extending compound. In one embodiment, a gold indicating electrode is coated with glucose oxidase and bovine serum albumin (BSA) and crosslinked with glutaraldehyde or a suberimidate compound, then coated with an outer membrane. A requirement may be that glucose must be able to permeate through the outer membrane. In one embodiment, the RMP and the enzyme are coated with a polymeric outer membrane. Oxygen permeability may not be necessary for the function of this type of sensor, but a degree of glucose permeability may be necessary. The outer membrane can be made of polyurethane, Nafion, poly(vinylpyridine), poly(vinylpyridine)-co-styrene, molecular weight cutoff polymeric membranes, silicone, hydrogels and many other materials that allow glucose permeation.

For example, a gold indicating electrode, coated with RMP and crosslinked glucose oxidase and polarized at 0-180 mV vs a Ag/AgCl reference electrode, is able to measure glucose with little or no interference from the preservatives used in insulin formulations. In contrast, the use of a platinum sensor, coated with crosslinked glucose oxidase and polarized at 400-750 mV, undergoes a very large oxidation current when exposed to phenolics. Furthermore, if such exposure lasts for more than a few minutes, the electrode is consistently poisoned by a dense layer of electropolymerized phenolic compound that prevents H₂O₂ and other common analytes from reaching the indicating electrode and being measured. This process results in a slow decay of the sensor signal.

One method of creating a continuous sensor built into the wall of an insulin infusion cannula is to laminate flexible thin metal films on the outer wall of a hollow tubular structure that serves as the conduit for the insulin formulation. This conduit (cannula) it may be made from a polymer, but can also be a metal in some designs. The polymer can be polyurethane, polyethylene, silicone, or many other polymerized compounds. In some cases, the cannula is flexible, but also can be rigid.

In some cases, it may be desirable to place a metallic foil between the thin film metal electrodes and the polymer that makes up the insulin delivery cannula. In some construction designs, the foil can enhance durability and fatigue resistance, though the presence of the foil is not necessary. The use of the term “foil” indicates a metal layer that is at least 2 micrometers (μm) in thickness, that is, thicker than the thin film layer typically deposited by sputtering, evaporation, printing or electroplating. The thin film layer is less than 2 micrometers.

All layers of the sensing catheter may be tightly adhered to the adjoining layers. One method of creating interfaces with good adhesion and good durability is the use a laminating press at high temperature and high pressure. Adhesives can be used to ensure that each layer stays applied to its neighboring layers.

The metal of which the foil is composed must be chosen carefully. In the case of an amperometric glucose sensor, the indicating electrode may be platinum, gold or carbon. Copper (which is commonly used as the foil for flexible electronic circuits), may not be suitable for use in a biosensor. Specifically, if there is concurrent physical contact between interstitial fluid, copper and platinum, a large galvanic current may occur as a result of a dissimilar metal junction. If a foil is used, titanium or gold can serve as the foil layer.

In some cases, there must be interconnect traces made of metal or conducting polymers that connect to the indicating electrode(s) and the reference electrode and terminate in a body-worn electronic sensor module which is in electrical continuity with the sensing catheter. The sensor module can contain a battery and a telemetry-enabled transceiver which transmits the electrochemical signals to a personal computer, mobile phone, or dedicated screen.

The glucose-sensing cannula can be used with insulin pumps that have a polymeric tube between the pump and the cannula. In such a case, the glucose-sensing cannula connects to the end of the tubing that terminates near the skin of the patient. In addition, the glucose-sensing cannula can also be used in pumps that have a smaller total footprint, e.g., those that omit the tubing wherein the pump containing the insulin reservoir is placed on or near the skin, and the cannula that passes through the skin is directly attached to the pump—these devices may be referred to as patch pumps. In such cases, the cannula for the patch pump may comprise the glucose-sensing cannula described above. Suitable insulin pumps include, but are not limited to, those marketed by Medtronic, Tandem Corp, Sooil, Abbott, Thermalin, Roche, Insulet, Ypsomed, CellNovo, CeQur, Dana Diabecare, EO Flow, and others.

A mediator-based glucose sensor may be placed in or on the outer wall of an insulin delivery cannula, as described by, for example, U.S. Pat. No. 10,780,222 (Ward et al), which is incorporated by reference herein in its entirety. For example, some of the materials and attributes of this sensing cannula, in vivo testing in swine, and in vitro tests of the device are described by (Ward, Heinrich, Breen et al, Diabetes Technol Ther 19:226-236, 2017), which is incorporated by reference herein in its entirety. By including an osmium complex (which includes an osmium-based mediator and a pyridine- or imidazole-based ligand), glucose oxidase, and a potential bias of less than 175 mV, this sensor may function in the direct presence of insulin and insulin analogs. However, when a conventional glucose sensor (requiring oxygen uptake and measuring the oxidation of hydrogen peroxide as the endpoint) was used in vitro and in vivo, there are two large types of artifacts observed. First, there is an immediate large rise in current, and second, there was a complete and permanent decay of the sensor's amperometric signal. This signal decay continues until the sensor was not even able to respond to a substantial concentration of hydrogen peroxide. Further testing indicates that these two artifacts were not caused by the insulin or insulin analog in the formulation. Instead, the artifacts are caused by the preservatives present in all insulin formulations, including phenol and a similar compound closely related to phenol, meta-cresol.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-80. (canceled)

81. A system for managing blood glucose in a subject, comprising:
an insulin delivery cannula comprising a hollow tube comprising a proximal end and a distal end, wherein the proximal end is in fluid communication with a source of an insulin or insulin analog formulation, wherein the distal end is configured to deliver the insulin or insulin analog formulation into a subcutaneous space of the subject;
a glucose sensor located no more than a pre-determined distance away from the distal end, wherein the glucose sensor is a continuous amperometric or coulometric glucose sensor comprising an indicating electrode and a reference electrode; and
an insulin infusion pump fluidically coupled to the proximal end of the insulin delivery cannula, wherein the insulin infusion pump is attached to the insulin delivery cannula either directly or via an intervening tube,
wherein the glucose sensor and the insulin delivery cannula are configured to be inserted into the subcutaneous space of the subject simultaneously by a single insertion device,
wherein a lumen of the insulin delivery cannula is contiguous with a lumen of tubing that emerges from the insulin infusion pump,
wherein electrical conductors for the indicating electrode and the reference electrode are disposed on a wall of the tubing or attached to a surface of the tubing, and establish an electrical connection with an electronic module located within a housing that encloses the insulin infusion pump.

82. The system of claim 81, wherein the insulin or insulin analog formulation comprises an excipient comprising a phenolic compound or cresol.

83. The system of claim 81, wherein the pre-determined distance is about 7 mm.

84. The system of claim 81, wherein the glucose sensor comprises an electrode layer comprising the indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase.

85. The system of claim 84, wherein the glucose sensor further comprises an insulating layer and a metal layer, wherein the insulating layer is coupled to the metal layer, and wherein the metal layer is coupled to the electrode layer.

86. The system of claim 84, wherein the redox mediator and the enzyme allow electron transfer from subcutaneous glucose to the indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode.

87. The system of claim 86, wherein the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the insulin or insulin analog formulation.

88. The system of claim 84, wherein the metal compound comprises a metal selected from the group consisting of: Osmium, Ruthenium, Palladium, Platinum, Rhodium, Iridium, Cobalt, Iron, and Copper.

89. The system of claim 81, wherein the glucose sensor is disposed on an outer wall of the hollow tube.

90. The system of claim 81, wherein the electrical conductors for the indicating electrode and the reference electrode are disposed on the wall of the tubing.

91. The system of claim 81, further comprising a transimpedance amplifier (TIA) operably connected to the glucose sensor, wherein the TIA is configured to convert an electrical current generated by the glucose sensor to a voltage value.

92. A method for managing blood glucose in a subject, comprising:
(a) obtaining a device for delivery of the insulin or insulin analog formulation and measurement of subcutaneous glucose concentration, wherein the device comprises:
(i) a hollow tube comprising a proximal end and a distal end, wherein the proximal end is in fluid communication with a source of an insulin or insulin analog formulation, wherein the distal end is configured to deliver the insulin or insulin analog formulation into a subcutaneous space of the subject;
(ii) a glucose sensor located no more than a pre-determined distance away from the distal end, wherein the glucose sensor is a continuous amperometric or coulometric glucose sensor comprising an indicating electrode and a reference electrode; and
(iii) an insulin infusion pump fluidically coupled to the proximal end of the insulin delivery cannula, wherein the insulin infusion pump is attached to the insulin delivery cannula either directly or via an intervening tube,
wherein a lumen of the insulin delivery cannula is contiguous with a lumen of tubing that emerges from the insulin infusion pump,
wherein electrical conductors for the indicating electrode and the reference electrode are disposed on a wall of the tubing or attached to a surface of the tubing;
(b) connecting the proximal end of the hollow tube to the source of the concentrated insulin or insulin analog formulation;
(c) establishing an electrical connection with an electronic module located within a housing that encloses the insulin infusion pump;
(d) inserting the glucose sensor and the distal end of the hollow tube of the insulin delivery cannula into the subcutaneous space of the subject simultaneously by a single insertion device; and
(e) simultaneously (1) delivering the insulin or insulin analog formulation into a subcutaneous space of the subject using the insulin infusion pump, and (2) measuring the subcutaneous glucose concentration of the subject.

93. The method of claim 92, wherein the insulin or insulin analog formulation comprises an excipient comprising a phenolic compound or cresol.

94. The method of claim 92, wherein the pre-determined distance is about 7 mm.

95. The method of claim 92, wherein the glucose sensor comprises an electrode layer comprising the indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase.

96. The method of claim 95, wherein the glucose sensor further comprises an insulating layer and a metal layer, wherein the insulating layer is coupled to the metal layer, and wherein the metal layer is coupled to the electrode layer.

97. The method of claim 95, wherein the redox mediator and the enzyme allow electron transfer from subcutaneous glucose to the indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode.

98. The method of claim 97, wherein the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the concentrated insulin or insulin analog formulation.

99. The method of claim 95, wherein the metal compound comprises a metal selected from the group consisting of: Osmium, Ruthenium, Palladium, Platinum, Rhodium, Iridium, Cobalt, Iron, and Copper.

100. The method of claim 92, wherein the glucose sensor is disposed on an outer wall of the hollow tube.

101. The method of claim 92, wherein the electrical conductors for the indicating electrode and the reference electrode are disposed on the wall of the tubing.

102. The method of claim 92, wherein a transimpedance amplifier (TIA) is operably connected to the glucose sensor, wherein the TIA is configured to convert an electrical current generated by the glucose sensor to a voltage value.
